(19) United States
(12) Patent Application Publication
(10) Pub. No.: US 2018/0031547 A1
(43) Pub. Date: Feb. 1, 2018

(54) DEVICES FOR IMMUNE-RELATED RISK ASSESSMENT

(71) Applicant: Maria D. F. S. Barbosa, Philadelphia, PA (US)

(72) Inventor: Maria D. F. S. Barbosa, Philadelphia, PA (US)

(21) Appl. No.: 15/631,152

(22) Filed: Jun. 23, 2017

Related U.S. Application Data
(63) Continuation-in-part of application No. 15/091,483, filed on Apr. 5, 2016.

(51) Int. Cl. G01N 33/543; G01N 33/531; G01N 33/53; A61B 5/145

(52) U.S. Cl. CPC G01N 33/54366; A61B 5/145; G01N 33/531; G01N 33/5302; A61B 5/00

(57) ABSTRACT

Portable devices for anti-drug antibody (ADA) testing, to estimate safety and/or efficacy of therapeutic agents are provided. These devices can be used to perform point of care risk assessment for various applications, including but not restricted to one or more of the following: selection of therapeutic drug for patient treatment; evaluation of the need to change therapeutic drug or to apply tolerance regimens; patient immune status prior and after vaccination; selection of patients for clinical trials; comparison of therapeutic drugs marketed for a given disease and also gene therapy; testing pre-existing antibodies as a risk factor; postmarketing surveillance of therapeutic drugs.

### CLAIM OF PRIORITY

This Divisional application claims priority to U.S. patent application Ser. No. 15/091,483, which claims priority to U.S. Provisional Patent Application No. 62/146,232 filed on Apr. 10, 2015, which are hereby incorporated by reference in their entirety.

### FIELD OF THE INVENTION

The present invention is related to devices used for evaluation antibody responses, and their applications. More specifically, the invention relates to devices for detecting anti-drug antibodies (ADAs), and estimating risk of adverse events and/or therapy failure in fields of oncology, autoimmune diseases (including but not restricted to diabetes, multiple sclerosis and rheumatoid arthritis), cardiovascular diseases, rare diseases, infectious diseases, and other diseases whose treatment comprises administration of a therapeutic entity and/or gene therapy.

### BACKGROUND OF THE INVENTION

A competent host immune system may mount antibody responses to a therapeutic agent (“drugs”) perceived as foreign, resulting in the formation of anti-drug antibodies—ADAs (Barbosa, M. D. F. S. and Smith, D. D. 2014 Drug Discov. Today. 19: 1897-1912; expressly incorporated by reference herein). Those antibody responses may be intentionally induced (as in the case of vaccines against pathogens such as bacteria or viruses), or may be an unintended and unanticipated effect of a therapeutic drug administration (Barbosa, M. D. F. S. and Smith, D. D. 2014 Drug Discov. Today. 19: 1897-1912; expressly incorporated by reference herein).

Surprisingly, in the case of many therapeutic drugs which have already been approved for marketing by regulatory authorities and are considered safe by healthcare professionals and patients, often ADA responses to them may result in adverse reactions of varying negative consequences, for example life-threatening IgE- or IgG-mediated anaphylaxis or anaphylactic shock (Barbosa, M. D. F. S. and Smith, D. D. 2014 Drug Discov. Today. 19: 1897-1912; expressly incorporated by reference herein). ADAs may also neutralize the drug effect, resulting in loss of efficacy. This is a problem that has been growing for no less than about eighteen years.

There is no standard point of care tests to readily detect ADAs. Hence, even in cases when alternative therapies are available, patients often continue to be treated with drugs unlikely to provide any therapeutic benefit. No less concerning are possible life-threatening adverse events due to ADAs, which may be pre-existing or develop during the course of therapy. It is very rare for doctors to test patients for ADAs after a drug has been approved and is being marketed (Kolata, G. The New York Times, May 15, 2017; expressly incorporated by reference herein). The available ADA assays require specialized technicians for execution, involve multiple steps and cumbersome sample collection and handling, and are typically difficult to reproduce from one laboratory to another, which precludes accurate comparisons.

Prior to this invention, which has been the result of much thought and experimentation, including trial and error, and is connected with a novel and holist approach to perform postmarketing risk assessment, there was no practical solution for the problem of health risks due to ADA incidence against marketed drugs (Barbosa and Smith, 2014 Drug Discovery Today Vol. 19, pages 1897-1912). This is despite several prior attempts at immunogenicity risk assessment for therapeutic drugs using theoretical pondering, including but not restricted to guidance from regulatory authorities (FDA Immunogenicity Assessment for Therapeutic Protein Products (2014); Shankar et al. Nature Biotechnology 2007 Vol. 25, pages 555-561; Jawa et al. Clinical Immunology 2013 Vol. 149, pages 534-555; all expressly incorporation by reference herein).

Described in this invention are point of care ADA testing devices that solve an urgent problem, presenting superior benefits while overcoming the deficiencies of previous assay.

An apparatus described by Plavina et al., which can detect anti-anti-VLA-4 antibodies (U.S. Pat. No. 9,377,458; expressly incorporated by reference herein) is a “bridging assay format”, which does not allow drug comparisons, and also presents many of the limitations of previous assays. For example, for detection each drug needs to be labeled separately, introducing sources of variability. In addition, Plavina et al. describes only a limited application for said assay.

Although prophylactic vaccines are intended to induce antibody responses, they prolific unnecessary use may also be problematic. In particular, there is no point of care testing to determine antibody levels against infectious diseases, prior to new vaccine shots to preserve immunity. This may lead to unnecessary vaccinations. It should be noted that in some instances antibodies can result in immune complexes involved with the etiology of diseases (Barbosa, M. D. F. S. and Smith, D. D. 2014 Drug Discov. Today. 19: 1897-1912; expressly incorporated by reference herein).

In mammals such as humans and mice, antibodies contain paired heavy and light polypeptide chains. Standard antibody structural units typically comprise a tetramer. Each tetramer is usually composed of two identical pairs of polypeptide chains, each pair having one “light” (typically having a molecular weight of about 25 kDa) and one “heavy” chain (typically having a molecular weight of approximately 50 kDa). “Isotype” as used herein is meant any of the subclasses of immunoglobulins. The known human antibody isotypes are IgG1, IgG2, IgG3, IgG4, IgA1, IgA2, IgM1, IgM2, IgD, and IgE.

Each antibody chain contains a variable and a constant region, as described above. The variable regions of the light and heavy chains are required for binding the target molecule (the antigen).

Host antibody responses against an antigen are typically polyclonal, comprising immunoglobulins that bind the antigen with various affinities and/or avidities. Hence, the assays used to detect antibody responses against therapeutic drugs are inherently qualitative, because there is no positive control antibody that would accurately represent all diverse antibodies in each of the samples collected from diverse sources and/or at various times following antigen exposure.

Additional problems solved by this invention are the cumbersome nature of collecting patient blood and shipping samples (commonly plasma or serum after blood processing) under special conditions to labs approved for ADA testing, and the lack of unified methodologies at such laboratories. In addition, in many instances laboratories offering those services are not even available and typically are not known by to physicians and/or patients (Kolata, G. The New York Times, May 15, 2017; expressly incorporated by reference herein). What follows is that there is an unmet need for devices to readily detect ADAs and to estimate risks. Such apparatus can have several utilities, including but not restricted to one or more of the following: stratification of patients likely to benefit from a given therapy, estimate risk of adverse events; comparison of similar products marketed for the same indication; tests during clinical trials; postmarketing surveillance.

ADA incidence against chronically administered products such as insulin and enzyme replacement therapies is also a concern. Even if the drug dosage is increased to compensate, the chronic administration may results in immune complexes not being cleared, leading to immune complex disease and/or other syndromes, including life-threatening anaphylaxis (Barbosa, M. D. F. S. and Smith, D. D. 2014 Drug Discov. Today 19: 1897-1912; Kolata, G. The New York Times, May 15, 2017; expressly incorporated by reference herein). In such cases, knowledge of ADA incidence and monitoring can provide an effective mechanism to evaluate risk and the need for tolerance induction regimens. There is no point of care testing for ADAs against insulin.

Another dreadful aspect of prescribing insulin to type 2 diabetics without knowing their ADA status (which is nowadays commonly done by healthcare professionals), is that ADAs developed against the drug may be cross-reactive with the endogenous insulin, and the patients may become dependent on insulin injections to sustain life. Currently there are approximately 30 million type 2 diabetics in the US, of which only about approximately 1.25 millions are diabetic type 1 (dependent on exogenous insulin to sustain life, that is). Very often type 2 diabetes patients can live healthy lives by seriously watching their diets, exercising and taking non-insulin medications. The ADA testing devices of the present invention can have an important role in risk assessment.

### BRIEF SUMMARY OF THE INVENTION

This invention is directed to portable ADA testing devices, to estimate safety and/or efficacy of therapeutic drugs for several applications, and/or to compare drugs used for treatment of the same diseases. ADAs resulting from vaccination against infectious agents can also be detected with said devices.

In one aspect, portable devices for ADA testing can be used by patients (self-test), or at a point of care such as a physician's office, or other healthcare facilities. In a further aspect, the portable ADA detection device can be used as a companion diagnostic.

In another aspect, the portable device for ADA detection contains an access code for a database. The information in the database can be with the device itself, or in a personal computer or other devices.

### BRIEF DESCRIPTION OF THE DRAWINGS

FIG. 1. Non-limiting example of a portable device to detect anti-drug antibodies (ADAs).

FIG. 2. Laboratory data obtained with a portable device that detects anti-drug antibodies (ADAs).

FIG. 3. Non-limiting example of a portable device for detection of both IgG and IgM, which uses the features described in FIG. 1 above, but with both labeled anti-IgG and labeled anti-IgM present in the matrix and samples running on parallel channels. Additional immunoglobulins can also be detected in additional channels.

FIG. 4. Portable devices as described in FIG. 1 and FIG. 3 above, in which a filter is added after position “1” for additional separation of cells and/or other debris. Alternatively, the sample pad can be adapted to perform a filtration step.

FIG. 5. Portable device for ADA detection (IgG and/or IgM and/or other ADA isotypes). The sample is placed in a sample pad at position “1”, and a handle may be placed on the opposite side (position “5). The ADAs in the test sample migrate towards a secondary antibody conjugated with a label, available at position “2” in the matrix. The sample ADAs are bound to the labeled secondary antibodies, and the complexes migrate towards position “3”, where they are captured by the test protein immobilized on the membrane, resulting in a signal. Position “4” may have a waste reservoir.

FIG. 6. Device for ADA detection. Sample is added to position “1” of the test strip, where the test antigen (+) is immobilized. Binding results in a signal.

FIG. 7. Portable device for ADA detection in which the sample is subjected to vertical flow, for example due to gravity (non-limiting example).

### DETAILED DESCRIPTION OF THE INVENTION

The present invention includes devices to detect host immune reactions, and to perform risk assessment for therapeutic entities. Devices enabling, but not restricted to, self-testing and/or testing at a point of care such as at physician's office, hospital or emergency room, or other point of care facilities, can be used to detect antibodies against therapeutic drugs or profilactic vaccines. That information regarding the presence or absence of antibodies against the drug can be used independently or combined with data from databases.

In order that the invention may be more completely understood, several definitions are set forth below. Such definitions are meant to encompass grammatical equivalents.

By “immunogenicity” as used herein is meant the ability of a protein or another substance or molecule to elicit a host's immune response.

By “tolerance” as used herein is meant immune tolerance to a protein or another substance or molecule, typically characterized by the lack of immune responses.

By “antibody” as used herein is meant a protein that binds an amino acid sequence or another molecular entity. In mammals such as humans and mice, antibodies contain paired heavy and light polypeptide chains. Each chain contains a variable and a constant region. The variable regions of the light and heavy chains are required for binding the target antigen.

By ‘ADA” or “anti-drug antibody” as used herein is meant antibody that bind to a protein or other molecular-entity or target antigen, whereas that protein or other molecular entity or antigen can be a therapeutic drug or other entity. In that sense, all antibodies are essentially “binding”.

By “antigen” as used herein is meant a substance that induces an immune response.

By “target antigen” as used herein is meant the molecule that is bound specifically by the variable region of a given antibody. A target antigen may be a protein, carbohydrate, lipid, oligonucleotide, or other chemical compound or entity.

By “antibody epitope” as used herein is meant the region of the target antigen that binds to the antibody variable region.

By “immunoglobulin (Ig)” herein is meant a protein consisting of one or more polypeptides substantially encoded by immunoglobulin genes. Immunoglobulins may have a number of structural forms, including but not limited to full length antibodies, antibody fragments, and individual immunoglobulin domains. Immunoglobulins include but are not limited to antibodies.

By “isotype” as used herein in regards to antibodies, is meant any of the subclasses of immunoglobulins defined by the chemical and antigenic characteristics of their constant regions. The currently known human immunoglobulin isotypes are IgG1, IgG2, IgG3, IgG4, IgA1, IgA2, IgM1, IgM2, IgD, and IgE.

By “IgG” as used herein is meant a polypeptide belonging to the class of antibodies that are substantially encoded by a recognized immunoglobulin gamma gene. In humans this class comprises IgG1, IgG2, IgG3, and IgG4. Also included are hybrids of IgG proteins in which amino acids for one IgG protein substituted for amino acids of a different IgG protein (e.g. IgG1/IgG2 hybrids).

By “amino acid” as used herein is meant one of the 20 naturally occurring amino acids or any non-natural analogues that may be present at a specific, defined position.

By “protein” herein is meant attached amino acids. The protein may be made up of naturally occurring amino acids and peptide bonds, or synthetic peptidomimetic structures, i.e. “analogs”, such as peptoids. Thus “amino acid”, or “peptide residue”, as used herein encompasses both naturally occurring and synthetic amino acids.

By “database” as used herein is meant a structured combination of information and/or data analyses, which can be accessed in one or more ways.

By “assay” as used herein is meant a procedure for testing samples.

By “ADA portable device” or “ADA device” or “ADA testing device” or “portable devices” as used herein is meant a portable device of the present invention, which allow for testing samples regarding the presence of ADA (ADAs).

By “PEGylation” as used herein is meant the addition of one or more polyethylene glycol (PEG) moiety by various means that may comprise the use of linkers.

By “variant protein”, “protein variant”, “variant polypeptide”, or “polypeptide variant” as used herein is meant a polypeptide sequence that differs from that of a parent polypeptide sequence by virtue of at least one amino acid modification. Variant polypeptide may refer to the polypeptide itself, a composition comprising the polypeptide, or the amino sequence that encodes it.

By “full length antibody” as used herein is meant the structure that constitutes the natural biological form of an antibody, including variable and constant regions, including one or more modifications. Alternatively, the antibodies can be a variety of structures, including, but not limited to, antibody fragments, monoclonal antibodies, bispecific antibodies, minibodies, domain antibodies, synthetic antibodies (sometimes referred to herein as “antibody mimetics”), chimeric antibodies, humanized antibodies, antibody fusions (sometimes referred to as “antibody conjugates”), and fragments of each. In certain variations, antibody may mean a protein consisting of one or more polypeptides substantially encoded by all or part of the recognized immunoglobulin genes. Antibody herein is meant to include full-length antibodies and antibody fragments, and may refer to a natural antibody from any organism, an engineered antibody, or an antibody generated by recombinant techniques for experimental, therapeutic, or other purposes.

By “labeled Antibodies” as used herein is meant antibodies that have the addition of one or more labels. For example, gold-labeled anti-host antibodies or other labels.

In another embodiment, proteins and/or other molecules can be labeled and used for generation of assay signal. In some embodiments, labels can be used in various forms to generate a detectable signal. The assay readout can be either the signal generated or inhibition of signal.

The term “labelling group” as used herein means any detectable label. In some embodiments, the labelling group is coupled to the antibody via spacer arms of various lengths to reduce potential steric hindrance. Various methods for labelling proteins are known in the art and may be used in performing the present invention. In general, currently known labels fall into a variety of classes, depending on the assay in which they are to be detected. For example: a) isotopic labels, which may be radioactive; b) magnetic labels; c) redox active moieties; d) optical dyes; e) enzymatic groups such as horseradish peroxidase, beta.-galactosidase, luciferase, alkaline phosphatase; f) biotinylated groups.

Specific labels can include but are not limited to optical dyes, including, but not limited to, chromophores, phosphors and fluorophores. Fluorophores can be either “small molecule” (chemical entity) or protein, or a combination.

By “fluorescent label” is meant any molecule that may be detected based on its fluorescent properties. Other appropriate optical dyes can also be used.

Appropriate proteinaceous labels also include, but are not limited to, green fluorescent protein (GFP) including from Renilla, Ptilosarcus, or Aequorea species, blue fluorescent protein, yellow fluorescent protein, luciferase, and beta galactosidase

Colloidal-gold, silver enhanced gold, blue latex bead and carbon black nanoparticles are labels known in the art that can also be utilized for the present invention Other labels capable of generating a suitable signal can also be used.

In another embodiment, novel labels discovered by any techniques, including but not restricted to genetic analysis of different species, or by any chemical, biochemical or other means, can be incorporated in assays used in the present invention, and are within the scope of its utility.

### Detection of Anti-Drug Antibodies (ADAs):

The present invention describes devices for detection of ADAs in body fluids (including but not restricted to blood and serum). Said devices are suitable for risk assessment. The described methods are not meant to constrain the present invention to any particular application or theory of operation. Rather, the provided methods are meant to illustrate generally that one or more techniques can be used to detect ADA against therapeutic drugs and vaccines, and can provide a useful means to evaluate risk of immune reactions. Clinical validation of the portable devices may include using them for tests with clinical samples, and comparison with other assays known in the art.

In one embodiment, the portable ADA testing device of the present invention may detect selected ADA isotypes. In another embodiment, the ADA assays of the present invention may comprise modifications to allow detection of all antibody isotypes. In another embodiment, the assays may also allow identification of the isotype species in the sample. The testing devices may be tailored to detect individual samples or multiple samples. In another embodiment, the portable device may be used for antibody epitope mapping.

By “lateral flow” or “lateral flow technology” or “lateral flow assay” as used herein is meant a technology or assay based on the principle that the test substance and/or reagents flow in one (or more than one) direction, and may result in detection of a test sub stance.

In a preferred embodiment, lateral flow is used in the ADA testing device. In another embodiment, vertical flow can be used.

In another preferred embodiment, the reactions of the portable ADA testing device can be performed without directional flow of reagents, samples or test substances.

None of the prior art utilizing lateral flow technology has identified the unifying devices and approach disclosed in embodiments of the present invention, for standardization of ADA measurements in the broad context of immunogenicity risk assessment. This is despite the fact that prior art on general principles of lateral flow technology dates of at least as early as 1971 (U.S. Pat. No. 3,620,677, which is incorporated herein by reference in its entirety). Additional non-limiting examples of patents disclosing lateral flow technology include the following US patent Numbers, all incorporated by reference in their entirety herein: U.S. Pat. Nos. 3,811,840; 3,888,629; 4,042,335; 4,168,146; 4,169,138; 4,258,001; 4,313,734; 4,235,601; 4,366,241; 4,348,207; 4,446,232; 4,435,504; 4,459,358; 4,503,143; 4,537,861; 4,594,327; 4,624,929; 4,703,017; 4,632,901; 4,756,828; 4,999,285; 4,654,309; 4,623,461; 4,806,311; 4,861,711; 4,868,108; 4,770,853; 4,803,170; 4,960,691; 5,030,558; 4,857,453; 4,855,240; 4,920,046; 4,963,468; 4,981,786; 5,006,474; 4,916,056; 4,956,302; 5,039,607; 5,079,174; 5,120,504; 5,075,078; 5,164,294; 5,141,850; 5,248,619; 5,356,782; 5,939,331; 6,485,982; 9,377,458.

In another embodiment, the portable device of the present invention can detect ADAs by generating a signal other than colorimetric, for example electrochemiluminescence, or when an electrical property is altered upon binding of ADA (U.S. Pat. No. 4,219,335; expressly incorporated by reference herein). Said electrical property includes one or more of the following: resistance; impedance; capacitance; electrical potential. Other methods to detect a signal upon binding of sample ADAs can be employed for the construction of a unifying ADA testing portable device, and that is included within embodiments of the present invention. Capture of the ADAs alters the electronic property of the nanotube transistors. Carbon nanotube biosensors are also included within embodiments of the present invention.

Modifications to increase sensitivity and accuracy of the assays may include but are not limited to, for example, optimization of the detection method and of sample collection and size, minimization of nonspecific background signal, matrix optimization, selection of time for assay development and signal reading. In another embodiment, modifications are made to improve biophysical properties of the regents used for the assay, including but not limited to stability, solubility, and oligomeric state.

In a preferred embodiment, the therapeutic drug can be an enzyme replacement therapy, an immunemodulator, an antibody, a therapeutic vaccine, an antimicrobial agent, or another agent administered to a patient for the purpose of eliciting a health-related benefit.

In another embodiment, the therapeutic drug is a biosimilar (Barbosa, M. D. F. S. and Smith, D. D. 2014. Drug Discov. Today 19: 1897-1912; expressly incorporated by reference herein). By “biosimilar’ as used herein is meant a therapeutic protein (biotherapeutic) similar to another one already marketed for which the patent has expired (the “reference product”).

In another embodiment, the therapeutic drug is a biobetter (Barbosa, M. D. F. S. and Smith, D. D. 2014 Drug Discov. Today 19: 1897-1912; expressly incorporated by reference herein). By “biobetter” as used herein is meant a newer version of a marketed biotherapeutic.

In another embodiment, the ADA testing device is connected to a database, which further allows assessment of risk of adverse events or likelihood of low drug efficacy. By “adverse event” as used herein is meant any undesirable experience (i.e., a bad side effect) associated with the use of a product.

In another preferred embodiment, the ADA testing device is used to evaluate postmarketing drug efficacy or safety. By “postmarketing” as used herein is meant after a therapeutic drug has received approval from a regulatory agency, for example the U.S. Food and Drug Administration (FDA) or the European Medicines Agency (EMA). In another embodiment, those postmarketing comparisons may also aid ranking of drugs approved for the same indication, including but not restricted to biosimilars and biobetters.

For the purpose of validation of the ADA-testing devices, they may be compared with one or more conventional assay used for a given drug, such as for example a radioimmunoassay to test for antibodies against insulin or another assay relevant for comparisons. The parameters tested may include but are not limited to factors such as sensitivity, robustness, inter and intra assay variation, precision, sensitivity, matrix interference, cut point determination, minimal required dilution, and drug inhibition of the assay (Barbosa, M. D. F. S. et al. 2006 Clin. Immunol. 118: 42-50; Barbosa, M. D. F. S. et al. 2012 J. Immunol. Methods 384:152-156; expressly incorporated by reference herein).

The ADA-testing devices may be further validated in clinical and/or preclinical studies. That validation may include comparison of data obtained with samples from the same humans or animal models, tested with an ADA-testing device and another assay known in the art or newly invented. Data obtained from those studies can be submitted to analysis and incorporated into databases. Other forms of device validation may also be used.

The ADA-testing devices can be used alone to provide information of the ADA positive or negative status or can be is used in conjunction with a database and with statistical analyses to infer the probability of safety or efficacy issues due to ADA responses.

In another preferred embodiment, the ADA-testing devices are used to compare therapeutic drugs used for the same indication.

The portable device can make the ADA information readily available to patients at home or at a point of care such as a physician's office, aiding therapeutic drug selection for use, and leading to corrective measures or the evaluation of the need to switch to another therapy. In cases when an alternative therapy is not an option (for example, for some currently used enzyme replacement therapies), the information obtained with the ADA testing device can guide the need for tolerance induction regimens.

In another embodiment, the ADA testing devices of the present invention can be used to guide selection of therapeutic drug dose. Therapeutic drug dose selection for humans is typically made during phase 1 clinical trials, using a limited number of human subjects. Pre-existing antibodies or ADAs that develops during the course of therapy can be an additional difficulty for selection of the correct dose of the corresponding therapeutic drug.

In another embodiment, the ADA testing device and corresponding database can be used to select patients for clinical trials, including but not restricted to clinical development of novel biotherapeutics, biosimilars or biobetters.

By “pre-existing antibody” as used herein, is meant an antibody against a therapeutic drug or other molecular entity that was present in the body of a human or animal prior to exposure to or administration of that therapeutic drug. In another preferred embodiment, the device of the present invention can be used to test pre-existing antibodies in humans or animals. Pre-existing ADAs may be indicative of risk of adverse reactions and/or low efficacy of the therapeutic drug.

In another embodiment, ADA testing with the device can indicate if a patient is immunized against one or more infectious agents, or if said patient needs vaccination.

### Clinical Use of ADA-Testing Devices and Related Databases:

The ADA-testing devices can be used within various therapeutic areas and animal disease models. Therapeutic areas in which this invention can be applied include but are not restricted to diabetes, cancer, inflammation, neurological diseases, cardiovascular disease, autoimmune diseases, antimicrobials, multiple sclerosis, and numerous rare diseases.

A “patient” for the purposes of the present invention includes both human and other animals, preferably mammals and most preferably humans.

The term “treatment” in the present invention is meant to include therapeutic treatment, as well as prophylactic (e.g. vaccines), or suppressive measures for a disease or disorder. “Treatment” also encompasses administration of a therapeutic drug after the appearance of the disease in order to ameliorate, control, or to eradicate the disease. Those “in need of treatment” include mammals already having the disease or disorder, as well as those prone to having the disease or disorder, including those in which the disease or disorder is to be prevented.

A variety of therapeutic drugs may be used for treatment of patients in diverse combinations, such use being described herein as “combination therapy”. For example, radiation and/or chemotherapy can be combined with a biotherapeutic anti-cancer drug, administered according to protocols commonly employed and known to the skilled artisan. In one embodiment, an ADA testing device is tailored to identify antibodies against the components of the combination therapy.

Included in embodiments of the present invention are companion diagnostic tests to identify patients who are likely to show a favorable clinical response to a therapeutic drug.

Furthermore, the present invention comprises prognostic tests performed on clinical samples such as blood, tissue and/or other samples. Such information may be used to identify patients for inclusion or exclusion in clinical trials, or to inform decisions regarding appropriate dosages and treatment regimens. Such information may also be used to select a therapeutic drug likely to provide superior therapeutic results.

### EXAMPLES

Non-limiting examples are provided below to illustrate the present invention. These examples are not meant to constrain the present invention to any particular recipe, therapeutic drug, application or theory of operation.

### Example 1

### Portable Devices for Anti-Drug Antibody (ADA) Testing at a Point of Care Facility or by a Patient

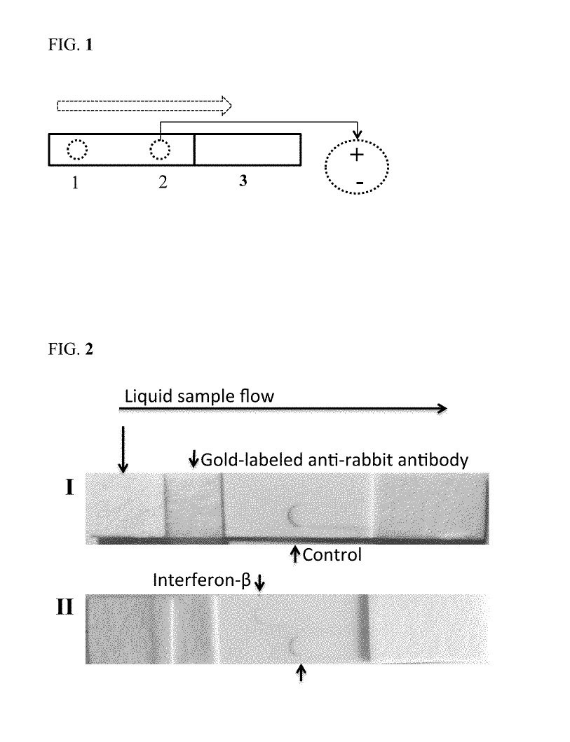

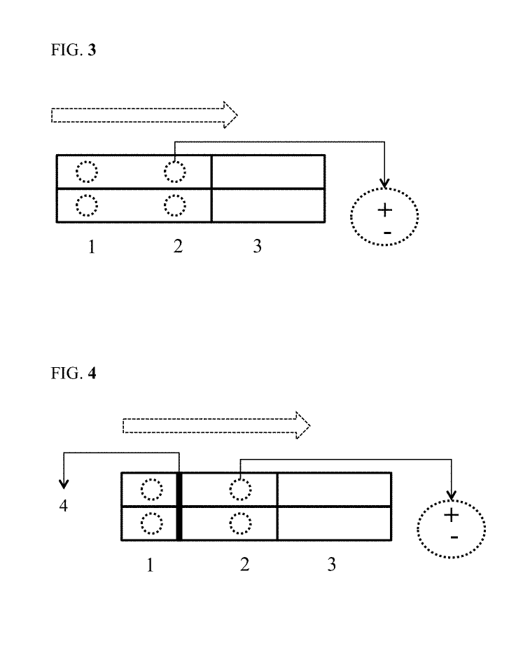

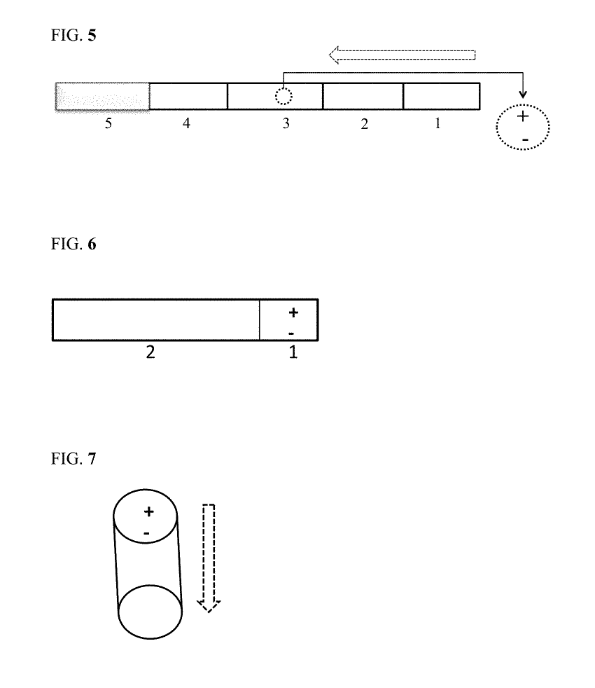

FIG. 1, FIG. 2, FIG. 3, FIG. 4, FIG. 5, FIG. 6, and FIG. 7 provide several non-limiting examples of portable devices that can be used for detecting ADA in body fluids and/or tissues. More specifically, in FIG. 1 the test-sample (blood, serum, plasma or other source) is added and at position “1”, and ADAs bind labeled anti-IgG present in the matrix. The IgG-anti-IgG complexes migrate by “lateral flow” towards the immobilized test protein and positive control (position “2”). When the sample reaches the test region, a defined pattern is displayed at position “2”: −, no IgG detected; ±, IgG detected. Position “3” has a waste reservoir or open end. A similar device can be used for IgM detection, in which case labeled anti-IgM is used to generate signal. A similar device can also be used for the detection of other ADAs besides IgG and IgM. Test components may be assembled in various formats, and they may also be mounted inside cases of different designs.

### Example 2

Laboratory Data Obtained with a Portable Device that Detects Anti Drug Antibodies (ADAs).

FIG. 2 shows a non-limiting example of the interior of a device used to test anti-IFN-β ADAs, consisting of a sample pad, followed by a conjugate pad, a membrane and an absorbent pad. In this case, the sample consisted of rabbit anti-human IFN-β ADAs; gold-labelled anti-rabbit was placed on the conjugate pad, and a control (anti-goat IgG) or human IFN-β immobilized on the membrane. As the sample flows towards the absorbent pad, the gold-labeled goat anti-rabbit antibodies bind to the constant region of the rabbit anti-IFN-β ADAs, which subsequently bind to the immobilized IFN-β, where the signal is generated. By changing the drug immobilized on the membrane and the labelled anti-ADA antibodies, ADAs against several other therapeutic drugs can be tested with a similar strategy. For instance, human insulin can be immobilized on a membrane and labelled anti-human antibodies placed on the conjugate pad, allowing for the detection of human anti-insulin antibodies. In another embodiment, the membrane can have immobilized distinct regions of the therapeutic protein, and/or peptides, allowing for antibody epitope mapping. This can help guide treatment selection. For example, if it is determined that the ADAs are directed toward one specific mutated region in a modified version of a therapeutic protein, the same biotherapeutic without that mutation may represent a more suitable treatment option. For point of care testing, the sample can be body fluids such as serum, plasma or blood. For patient self-testing the sample can be, for example, one blood drop obtained using a lancet. In another embodiment, the portable device can contain a code or another means of allowing access to a database related to the therapeutic drug or related data.

1. A portable device for testing anti-drug antibodies (ADAs) in tissues and body fluids, comprising:
a. A support that can be hand-held or rest on a surface;
b. Labeled entities that can bind to a constant region of ADAs, wherein the labeled entities bound to ADAs flow towards one or more target antigens; and
c. One or more target antigens immobilized on the device, wherein binding of a target antigen to ADAs bound to the labeled entities results in a detectable signal.

2. The device of claim 1 allowing access to a database.

3. The device of claim 2, wherein the database is on the device.

4. The device of claim 2, wherein the database is accessed on a separate location.

5. The device of claim 1, wherein said device is based on lateral-flow technology, and can detect immunoglobulins selected from the group consisting of IgG, IgM, IgE, immunoglobulin isotypes.

6. The device of claim 5, wherein said device can detect more than one immunoglobulin isotype.

7. The device of claim 1, wherein said device is based on vertical flow technology, and can detect immunoglobulins selected from the group consisting of IgG, IgM, IgE, immunoglobulin isotypes.

8. The device of claim 7, wherein said device can detect more than one immunoglobulin isotype.

9. The device of claim 1, using a matrix for localized reactions, wherein said device can detect immunoglobulins selected from the group consisting of IgG, IgM, IgE, immunoglobulin isotypes.

10. The device of claim 9, wherein said device can detect more than one immunoglobulin isotype.

11. The device of claim 1, wherein one or more target antigens are immobilized at different positions on the device.

12.

13.

14.

15.

16.

17.

18.

19. The device of claim 1, wherein the target antigens are from the group consisting of an entire therapeutic drug, specific regions of a therapeutic drug, and drug degradation products.

20. The device of claim 1, wherein the target antigens are regions of proteins or peptides.

21. The device of claim 1, wherein the target antigens are from the group consisting of polyethylene glycol, chemical compounds, and chemical entities.

22. the device of claim 1, wherein the target antigens are oligonucleotides.

23. The device of claim 1, wherein the targets antigens are lipids.

24. The device of claim 1, wherein the target antigens are carbohydrates.

25. The device of claim 1, wherein the target antigens are from polyclonal or monoclonal antibodies.

26. The device of claim 1, wherein the target antigens are from vaccines.
